(19) United States
(12) Patent Application Publication
(10) Pub. No.: US 2025/0099953 A1
(43) Pub. Date: Mar. 27, 2025

(54) COLLECTOR, CELL SORTING DEVICE, AND METHOD FOR CELL COLLECTION

(71) Applicant: Suzhou Labyrinth Biotech Co., Ltd., Suzhou (CN)

(72) Inventors: Lu CHEN, Suzhou (CN); Yan ZHAO, Suzhou (CN); Jianfei HE, Suzhou (CN); Yansheng ZHANG, Suzhou (CN); Boyi YAO, Suzhou (CN)

(73) Assignee: Suzhou Labyrinth Biotech Co., Ltd., Suzhou, JI (CN)

(21) Appl. No.: 18/291,387

(22) PCT Filed: Aug. 29, 2023
(86) PCT No.: PCT/CN2023/115607

§ 371 Date: Jan. 23, 2024

(30) Foreign Application Priority Data
Nov. 1, 2022 (CN) 202211356520.7

(51) Int. Cl. B01L 3/00; C12M 1/00

(52) U.S. Cl. CPC B01L 3/502707; C12M 47/04; B01L 2200/027; B01L 2300/041; B01L 2300/0819

(57) ABSTRACT

The present disclosure relates to a collector, a cell sorting device, and a method for cell collection. The collector comprises: a housing which is internally provided with a mixing chamber and a loading chamber and which is provided with a filling hole in communication with the mixing chamber; a microfluidic chip arranged in the housing; and a collection vessel detachably connected to the housing. A target cell solution separated by the microfluidic chip can flow into the collection vessel. An inlet of the microfluidic chip, the loading chamber, and the collection vessel are selectively in communication with the mixing chamber through a communicating structure, respectively. After the microfluidic chip discharges the target cell solution separated for a first time into the collection vessel, the collection vessel is in communication with the mixing chamber through the communicating structure, and a negative pressure is inputted into the mixing chamber through the filling hole, such that the target cell solution in the collection vessel is sucked into the mixing chamber. Afterwards, the microfluidic chip is in communication with the mixing chamber through the communicating structure, and a positive pressure is inputted into the mixing chamber through the filling hole, such that the target cell solution in the mixing chamber is pressurized into the microfluidic chip. In this way, secondary separation is achieved through the microfluidic chip.

### TECHNICAL FIELD

The present disclosure relates to the field of medical instrument technology, and more particularly, to a collector, a cell sorting device, and a method for cell collection.

### BACKGROUND

Cell sorting belongs to a technology for separating target cells from samples, and the process of cell sorting needs to be achieved by use of a collector.

The collector includes a microfluidic chip and a collection vessel. After the samples are injected into the microfluidic chip, the microfluidic chip can separate out the target cells and collect the target cells with the collection vessel. A patent with publication number CN 112159752 A discloses a cell separation apparatus, which includes a sample input apparatus, a microfluidic chip box, and a sample collection apparatus. The sample collection apparatus includes a support, a sample collection holder and a liquid waste collection box arranged inside the support. The sample collection holder is internally provided with a sample collection tube. The sample input apparatus is used to inject the samples into the microfluidic chip of the microfluidic chip box. The sample collection tube is used to collect the target cells discharged by the microfluidic chip in the microfluidic chip box. Research results of the existing technologies indicate that secondary separation of the samples can better remove background impurities and obtain a solution of the target cells with higher purity. However, if the above-mentioned cell separation apparatus needs to perform secondary separation on the solution of the target cells in the sample collection apparatus, the sample collection tube must be first removed from the sample collection holder, then the solution of the target cells in the sample collection tube is injected into the sample input apparatus, and then the solution is injected into the microfluidic chip through the sample input apparatus. Therefore, the process of the secondary separation of the sample is complex, which leads to a lower efficiency of cell sorting and may cause contamination to the samples in the operation process.

Therefore, there is an urgent need for a collector to solve the aforementioned technical problems.

### SUMMARY

One objective of the present disclosure is to propose a collector for collecting target cell solutions after secondary separation.

To achieve the above objective, a first aspect of the present disclosure provides a collector, which includes:
a housing internally provided with a mixing chamber and a loading chamber, where the housing is provided with a filling hole in communication with the mixing chamber;
a microfluidic chip arranged on the housing; and
a collection vessel detachably connected to the housing, where the target cell solution separated by the microfluidic chip can flow into the collection vessel.

An inlet of the microfluidic chip, the loading chamber, and the collection vessel are selectively in communication with the mixing chamber through a communicating structure, respectively.

Alternatively, the communicating structure includes a loading chamber connection hose, a chip connection hose and a collection vessel connection hose.

The loading chamber is in communication with the mixing chamber through the loading chamber connection hose, the inlet of the microfluidic chip is in communication with the mixing chamber through the chip connection hose, and the collection vessel is in communication with the mixing chamber through the collection vessel connection hose.

The housing is provided with avoidance ports allowing the loading chamber connection hose, the chip connection hose, and the collection vessel connection hose to be at least partially exposed outside.

Alternatively, the housing is provided with a chip connection hole, one end of the chip connection hole is in communication with a lower end of the mixing chamber, and other end of the chip connection hole is connected to the chip connection hose.

Alternatively, the housing is provided with a loading chamber connection hole and a collection vessel connection hole near an upper end of the loading chamber, the loading chamber connection hose is in communication with the mixing chamber through the loading chamber connection hole, and the collection vessel connection hose is connected to the mixing chamber through the collection vessel connection hole.

Alternatively, an outer lateral wall of the housing is provided with holding slits configured to respectively hold the loading chamber connection hose, the chip connection hose and the collection vessel connection hose, and one of the holding slits is in communication with one of the avoidance ports.

Alternatively, one lateral wall of the housing includes an inner plate and a cover plate, the holding slit is provided on a side of the inner plate near the cover plate, and the avoidance port runs through the cover plate.

Alternatively, a holding groove is provided on an outer lateral wall of the housing, and the collection vessel is arranged in the holding groove.

Alternatively, the collector also includes a cover body, which can cover the collection vessel, where a clamping slot is provided on the outer lateral wall of the housing, and the cover body is seated in the clamping slot.

Another objective of the present disclosure is to provide a cell sorting device for collecting target cell solutions after secondary separation.

To achieve this objective, a second aspect of the present disclosure adopts the following technical solutions,

A cell sorting device includes the collector as described above.

Another objective of the present disclosure is to provide a cell sorting method for collecting the target cell solutions after secondary separation.

To achieve this objective, a third aspect of the present disclosure adopts the following technical solutions.

A method for cell collection uses the collector or the cell sorting device as described above, and the method for cell collection includes the following steps.

When it is required to inject a solution in the mixing chamber into the microfluidic chip, the mixing chamber is in communication with the microfluidic chip through the communicating structure, and a positive pressure is inputted into the mixing chamber through the filling hole.

When it is required to inject the solution in the loading chamber or the collection vessel into the mixing chamber, the mixing chamber is in communication with the loading chamber or the collection vessel through the communicating structure, and a negative pressure is inputted into the mixing chamber through the filling hole.

As can be seen from the above technical solutions provided by the present disclosure, after the microfluidic chip discharges the target cell solution separated for a first time into the collection vessel, the communicating structure enables the collection vessel to be in communication with the mixing chamber, and a negative pressure is inputted into the mixing chamber through the filling hole, such that the target cell solution in the collection vessel is sucked into the mixing chamber. Afterwards, the communicating structure enables the microfluidic chip to be in communication with the mixing chamber, and a positive pressure is inputted into the mixing chamber through the filling hole, such that the target cell solution in the mixing chamber is pressurized into the microfluidic chip. In this way, secondary separation is achieved through the microfluidic chip.

### BRIEF DESCRIPTION OF THE DRAWINGS

FIG. 1 is a structural schematic diagram of a collector according to an embodiment of the present disclosure;

FIG. 2 is a structural schematic diagram of the interior of a housing according to an embodiment of the present disclosure;

FIG. 3 is a cross-sectional view of the collector according to an embodiment of the present disclosure;

FIG. 4 is a structural schematic diagram of the collector after removal of the cover plate and the hose according to an embodiment of the present disclosure; and

FIG. 5 is a structural schematic diagram of the collector from another perspective according to an embodiment of the present disclosure.

Reference signs in the figures:
1, housing; 11, mixing chamber; 12, loading chamber; 13, filling hole; 14, chip connection hole; 15, loading chamber connection hole; 16, collection vessel connection hole; 17, inner plate; 171, holding slit; 18, cover plate; 19, liquid waste chamber; 101, clamping slot; 102, plug-in slot; 103, first sealing ring; 104, second sealing ring; 105, holding groove;
2, microfluidic chip;
3, collection vessel;
4, communicating structure; 41, loading chamber connection hose; 42, chip connection hose; 43, collection vessel connection hose;
5, avoidance port; 6, upper cover; and 7, cover body.

### DETAILED DESCRIPTION

Technical solutions of the present disclosure are further described below through embodiments with reference to the accompanying drawings. It is to be understood that the embodiments set forth herein are merely intended to interpret the present disclosure instead of limiting the present disclosure. In addition, it is also to be noted that for ease of description, drawings merely show parts related to the present disclosure instead of all parts.

In the present disclosure, certain directional words are defined. Without any contrary explanation, the directional words such as “up”, “down”, “left”, “right”, “inside”, “outside”, are used for the purpose of facilitating understanding and therefore do not constitute a limitation on the protection scope of the present disclosure.

In the present disclosure, unless specified or limited otherwise, a structure in which a first feature is “on” or “below” a second feature may include an embodiment in which the first feature is in direct contact with the second feature, and may also include an embodiment in which the first feature and the second feature are not in direct contact with each other, but are contacted via an additional feature therebetween. Furthermore, a first feature “on,” “above,” or “on top of” a second feature may include an embodiment in which the first feature is right or obliquely “on,” “above,” or “on top of” the second feature, or just means that the first feature is at a horizontal height more than that of the second feature. A first feature “below,” “under,” or “underneath” a second feature may include an embodiment in which the first feature is right or obliquely “below,” “under,” or “underneath” the second feature, or just means that the first feature is at a height less than that of the second feature.

In the description of the present disclosure, it is to be noted that unless specified or limited otherwise, terms such as “linking”, “connecting” and “fixation” should be understood in a broad sense, which may be, for example, a fixed connection, a detachable connection or integrated connection, a mechanical connection or an electrical connection, a direct connection or indirect connection by means of an intermediary, an internal communication between two components or an interaction relationship between two components. For those of ordinary skills in the art, specific meanings of the above terms in the present disclosure may be understood based on specific circumstances.

This embodiment provides a collector for separating and collecting target cells to improve cell sorting efficiency and reduce sample contamination probability.

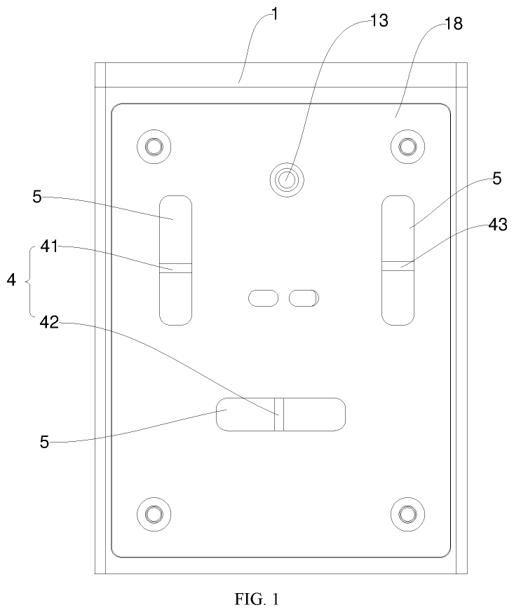

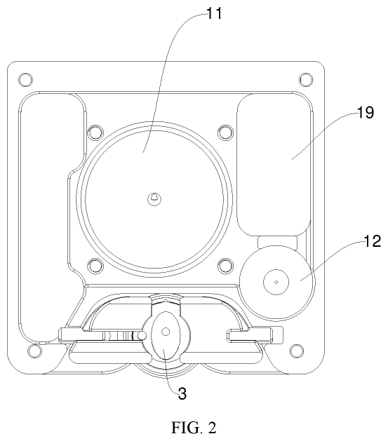

As shown in FIGS. 1 and 2, the collector provided in this embodiment includes a housing 1, a microfluidic chip 2, and a collection vessel 3. The housing 1 is internally provided with a mixing chamber 11 and a loading chamber 12, and the housing 1 is provided with a filling hole 13 in communication with the mixing chamber 11. The microfluidic chip 2 is arranged in the housing 1, and is used to separate target cells in a sample. Specific structures and operating principles of the microfluidic chip 2 belong to the existing technologies, and thus the same will not be repeated here. The collection vessel 3 is detachably connected to the housing 1, and a target cell solution separated by the microfluidic chip 2 can flow into the collection vessel 3, which may be a test tube or a centrifuge tube, etc. An inlet of the microfluidic chip 2, the loading chamber 12, and the collection vessel 3 are selectively in communication with the mixing chamber 11 through a communicating structure 4, respectively.

The filling hole 13 is in communication with the mixing chamber 11, and a cell sorting device may inject a rinse solution and/or a diluent into the mixing chamber 11 through the filling hole 13. Furthermore, positive or negative pressure may be inputted into the mixing chamber 11 through the filling hole 13. That is, gas is filled into the mixing chamber 11 such that a gas pressure in the mixing chamber 11 is greater than atmospheric pressure to form the positive pressure, or the gas is extracted from the mixing chamber 11 such that the gas pressure in the mixing chamber 11 is less than the atmospheric pressure to form the negative pressure.

When it is required to inject a solution in the mixing chamber 11 into the microfluidic chip 2, the mixing chamber 11 is in communication with the microfluidic chip 2 through the communicating structure 4, and the positive pressure is inputted into the mixing chamber 11 through the filling hole 13 such that the solution in the mixing chamber 11 is pressurized into the microfluidic chip 2. When it is required to inject the solution in the loading chamber 12 or the collection vessel 3 into the mixing chamber 11, the mixing chamber 11 is in communication with the loading chamber 12 or the collection vessel 3 through the communicating structure 4, and the negative pressure is inputted into the mixing chamber 11 through the filling hole 13 such that the solution in the loading chamber 12 or the collection vessel 3 is sucked into the mixing chamber 11.

After the microfluidic chip 2 discharges the target cell solution separated for the first time into the collection vessel 3, the communicating structure 4 enables the collection vessel 3 to be in communication with the mixing chamber 11, and the negative pressure is inputted into the mixing chamber 11 through the filling hole 13, such that the target cell solution in the collection vessel 3 is sucked into the mixing chamber 11. Afterwards, the communicating structure 4 enable the microfluidic chip 2 to be in communication with the mixing chamber 11, and the positive pressure is inputted into the mixing chamber 11 through the filling hole 13, such that the target cell solution in the mixing chamber 11 is pressurized into the microfluidic chip 2. In this way, secondary separation is achieved through the microfluidic chip 2.

Furthermore, the loading chamber 12 can provide samples to the mixing chamber 11 by means of the communicating structure and the cooperation between the positive and negative pressures inside the mixing chamber 11 with the collector provided in this embodiment. After the samples are diluted in the mixing chamber 11, the diluted samples can enter the microfluidic chip 2, thereby achieving the loading operation of the microfluidic chip 2 without the need of additional loading apparatus. In this way, structures of the collector are simplified, and costs of the collector are reduced.

As shown in FIG. 1, alternatively, the communicating structure 4 includes a loading chamber connection hose 41, a chip connection hose 42, and a collection vessel connection hose 43. The loading chamber 12 is in communication with the mixing chamber 11 through the loading chamber connection hose 41, the inlet of the microfluidic chip 2 is in communication with the mixing chamber 11 through the chip connection hose 42, and the collection vessel 3 is in communication with the mixing chamber 11 through the collection vessel connection hose 43. The housing 1 is provided with avoidance ports 5 allowing the loading chamber connection hose 41, the chip connection hose 42, and the collection vessel connection hose 43 to be at least partially exposed outside. Alternatively, one avoidance port 5 corresponds to one hose to operate the corresponding hose. Specifically, the avoidance port 5 is passed through manually or with a component such as a gripper on the cell sorting device, and then the loading chamber connection hose 41, the chip connection hose 42 or the collection vessel connection hose 43 are pressed to prevent the solution from passing through; and when the loading chamber connection hose 41, the chip connection hose 42 or the collection vessel connection hose 43 is released, the solution is allowed to pass through.

Alternatively, the loading chamber connection hose 41 may be in communication with a lower end of the loading chamber 12 to minimize or avoid residual solution in the loading chamber 12. The collection vessel connection hose 43 can be in communication with a lower end of the collection vessel 3 to minimize or avoid residual solution in the collection vessel 3.

In another embodiment, the communicating structure 4 may include a pipeline and a valve connected to the pipeline, where on or off of the pipeline can be controlled by means of the valve. The pipeline may be a rigid pipe with a fixed shape, such as a pipeline made of plastic or metal. As a manual valve such as a rotary valve or a passage valve, the valve is positioned in the avoidance port 5, such that the valve can be opened or closed manually or through the gripper on the cell sorting device; alternatively, the valve is an automatic control valve such as an electromagnetic valve, and on or off of the valve can be controlled by means of a control system, such that the inlet of the microfluidic chip 2, the loading chamber 12, and the collection vessel 3 are selectively in communication with the mixing chamber 11, respectively.

As shown in FIG. 2, alternatively, the housing 1 may also be internally provided with a liquid waste chamber 19, which is in communication with a liquid waste outlet of the microfluidic chip 2 to discharge liquid waste into the liquid waste chamber 19.

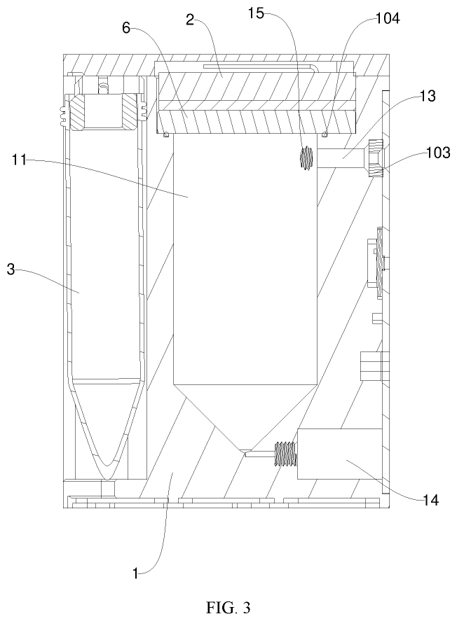

As shown in FIG. 3, the microfluidic chip 2 may be positioned above the liquid waste chamber 19 and the collection vessel 3, to facilitate discharge of the liquid waste and the target cell solution.

To communicate the chip connection hose 42 to the mixing chamber 11, alternatively, the housing 1 is provided with a chip connection hole 14, and one end of the chip connection hole 14 is in communication with a lower end of the mixing chamber 11 to drain out the solution in the mixing chamber 11, and other end of the chip connection hole 14 is connected to the chip connection hose 42 such that the mixing chamber 11 is in communication with the microfluidic chip 6.

Alternatively, the lower end of mixing chamber 11 has a conical structure to avoid residual solution at the bottom of the mixing chamber 11.

Alternatively, alternatively, an inner wall of the filling hole 13 is connected with a first sealing ring 103, to avoid leakage when the mixing chamber 11 is filled with gases or liquids. An upper end of the liquid waste chamber 19 is provided with an upper cover 6, and a second sealing ring 104 is provided between the upper cover 6 and the liquid waste chamber 19 to maintain the positive or negative pressure inside the liquid waste chamber 19.

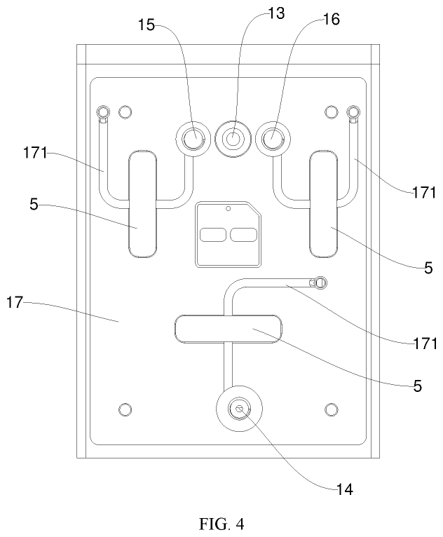

As shown in FIG. 3 and FIG. 4, the housing 1 is provided with a loading chamber connection hole 15 and a collection vessel connection hole 16, where the loading chamber connection hose 41 is in communication with the mixing chamber 11 through the loading chamber connection hole 15, and the collection vessel connection hose 43 is connected to the mixing chamber 11 through the collection vessel connection hole 16. Alternatively, both the loading chamber connection hole 15 and the collection vessel connection hole 16 are arranged near an upper end of the mixing chamber 11. After the solution in the mixing chamber 11 is filled in place, both the loading chamber connection hole 15 and the collection vessel connection hole 16 can be positioned on an upper side of a liquid level such that the solution in the mixing chamber 11 can be prevented from entering the loading chamber connection hose 41 and the collection vessel connection hose 43 uncontrollably.

Alternatively, the filling hole 13 is also arranged near the upper end of the mixing chamber 11, such that when a solution is filled into the mixing chamber 11, the gas above the solution can be extracted or a pressure can be applied above of the solution. Furthermore, the filling hole 13, the loading chamber connection hole 15, and the collection vessel connection hole 16 are positioned at a same height.

As shown in FIG. 4, an outer lateral wall of the housing 1 is provided with holding slits 171 configured to respectively hold the loading chamber connection hose 41, the chip connection hose 42 and the collection vessel connection hose 43, and one of the holding slits 171 is in communication with one of the avoidance ports 5. The holding slits 171 may limit a position of a hose, such that the hose is prevented from dead bending, which can in turn lead to poor flow of the solution.

As shown in FIGS. 1 and 4, one lateral wall of the housing 1 includes an inner plate 17 and a cover plate 18, the holding slit 171 is provided on a side of the inner plate 17 near the cover plate 18, and the avoidance port 5 runs through the cover plate 18. The avoidance port 5 may also extend to the inner plate 17, and the avoidance port 5 may or may not run through the inner plate 17, as long as the hose can smoothly extend from the holding slit 171 to the avoidance port 5. The cover plate 18 can protect the hose from accidental contact. It can be understood that the inner plate 17 and the cover plate 18 are arranged along a wall thickness direction of the housing 1. The inner plate 17 and the cover plate 18 may be connected by screws, or by clamps, and modes of specific connection between the inner plate 17 and the cover plate 18 are not limited.

The chip connection hole 14, the loading chamber connection hole 15, and the collection vessel connection hole 16 may all be provided on the inner plate 17.

The holding slit 171 and the avoidance port 5 are provided on the same lateral wall of the housing 1 to facilitate manipulation and control of a plurality of hoses by the cell sorting device.

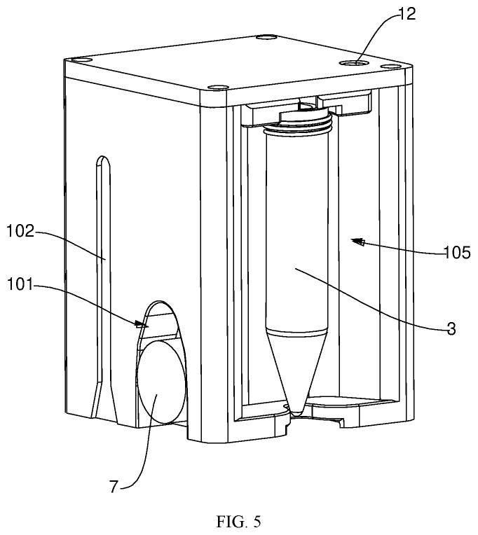

As shown in FIG. 5, an opening of the loading chamber 12 may be positioned at an upper end of the housing 1, making it convenient for the user to load samples into the loading chamber 12.

The outer lateral wall of the housing 1 is provided with a holding groove 105, and the collection vessel 3 is arranged in the holding groove 105. Alternatively, the holding groove 105 is provided on an outer side of the outer lateral wall to facilitate opening and placing the holding groove 105. The holding groove 105 may be internally provided with a socket, and an upper or lower end of the collection vessel 3 may be inserted into the socket to fix the collection vessel 3. In other optional embodiments, the collection vessel 3 may also be detachably connected to the holding groove 105 by other means, which are not limited here.

The collector also includes a cover body 7, which can cover the collection vessel 3, where a clamping slot 101 is provided on the outer lateral wall of the housing 1, and the cover body 7 is seated in the clamping slot 101.

A lateral wall of the housing 1 is provided with a plug-in slot 102 for fixing the collector. Alternatively, the plug-in slot 102 extends along a vertical direction, and a guide structure may also be arranged at a lower end of the plug-in slot 102 to facilitate entering of a protrusion into the plug-in slot 102.

Operating process of using the collector provided in this embodiment is as follows.
1. Loading: samples are loaded into the loading chamber 12, and the loading chamber connection hose 41, the chip connection hose 42, and the collection vessel connection hose 43 are all in an off state;
2. Injecting a rinse solution: the rinse solution is injected into the mixing chamber 11 through the filling hole 13;
3. Rinsing the microfluidic chip 2: the chip connection hose 42 is in communication with the microfluidic chip 2 and the mixing chamber 11, and a positive pressure is inputted into the mixing chamber 11 through the filling hole 13, such that the rinse solution in the mixing chamber 11 is pressurized into the microfluidic chip 2, and the solution in the microfluidic chip 2 is discharged to the liquid waste chamber 19 or to the liquid waste chamber 19 and the collection vessel 3; the chip connection hose 42 is disconnected;
4. Injecting a diluent: the diluent is injected into the mixing chamber 11 through the filling hole 13;
5. Diluting the samples: the loading chamber connection hose 41 is in communication with the loading chamber 12 and the mixing chamber 11, and a negative pressure is inputted into the mixing chamber 11 through the filling hole 13, such that the samples in the loading chamber 12 are sucked into the mixing chamber 11, thereby obtaining a diluted sample solution; the loading chamber connection hose 41 is disconnected;
6. Separating the samples for the first time: the chip connection hose 42 is in communication with the microfluidic chip 2 and the mixing chamber 11, and a positive pressure is inputted into the mixing chamber 11 through the filling hole 13, such that the sample solution in the mixing chamber 11 is pressurized into the microfluidic chip 2, the microfluidic chip 2 separates the sample solution and discharges the target cell solution obtained from the first separation into the collection vessel 3, while the remaining solution is discharged into the liquid waste chamber 19; and the chip connection hose 42 is disconnected;
7. The sample obtained from the first separation flows back to the mixing chamber 11: the collection vessel connection hose 43 is in communication with the collection vessel 3 and the mixing chamber 11, and a negative pressure is inputted into the mixing chamber 11 through the filling hole 13, such that the target cell solution in the collection vessel 3 flows back to the mixing chamber 11, to prepare for the secondary separation of the samples; and the collection vessel connection hose 43 is disconnected;
8. Diluting the samples separated for the first time: the diluent is injected into the mixing chamber 11 through the filling hole 13 to dilute the target cell solution in the mixing chamber 11;
9. Separating the samples for the second time: the chip connection hose 42 is in communication with the microfluidic chip 2 and the mixing chamber 11, and a positive pressure is inputted into the mixing chamber 11 through the filling hole 13, such that the sample solution in the mixing chamber 11 is pressurized into the microfluidic chip 2, the microfluidic chip 2 separates the sample solution and discharges the target cell solution obtained from the secondary separation into the collection vessel 3, while the residual solution is discharged into the liquid waste chamber 19;
10. The collection vessel 3 is taken out, and the cover body 7 is also taken out and covered on the collection vessel 3.

The target cells of the collector provided in this embodiment are obtained by inputting the pressure in the mixing chamber 11 and controlling on/off of the communicating structure 4, thereby achieving the secondary separation of the sample, which greatly simplifies the operation process and improves the sorting efficiency. During the secondary separation, the samples are not taken out from the collector, thus avoiding secondary contamination of the samples.

This embodiment also provides a cell sorting device, which includes the collector. The cell sorting device may also include a device body, which may also be provided with a plug-in block plugged into the plug-in slot 102 to stably install the collector on the device body.

Alternatively, the collector provided in this embodiment may be a disposable consumable pack. As an integral structure, the collector only needs to be installed on the device body for cell separation, which is convenient for operation and higher in operating efficiency. From the sample injection to the separation of the target cell solution obtained by the secondary separation, steps are all performed on the collector. For the cell sorting device, there is no other material loss, so there is no need to disassemble a plurality of consumable packs, which further improves the operating efficiency.

Alternatively, the cell sorting device also includes a filling component that can be inserted into the filling hole, where the filling component is configured to fill the mixing chamber 11 with the rinse solution, the diluent or gases, or to extract at least a portion of the gases from the mixing chamber 11.

It can be understood that number of the mixing chambers 11, the loading chambers 12, the microfluidic chips 2, the collection vessels 3, and the liquid waste chambers 19 provided in this embodiment may be one or more. The number of the mixing chambers 11, the loading chambers 12, the microfluidic chips 2, the collection vessels 3, and the liquid waste chambers 19 may be equal or may be not equal, and may be selected according to needs, which are not limited in this embodiment and are all within the scope of protection of the present disclosure.

This embodiment also provides a method for cell collection utilizing the aforementioned collector or cell sorting device. The method for cell collection includes:
communicating the mixing chamber 11 with the microfluidic chip 2 through the communicating structure 4, and inputting a positive pressure into the mixing chamber 11 through the filling hole 13, when it is required to inject the solution in the mixing chamber 11 into the microfluidic chip 2; and
communicating the mixing chamber 11 with the loading chamber 12 or the collection vessel 3 through the communicating structure 4, and inputting a negative pressure into the mixing chamber 11 through the filling hole 13, when it is required to inject the solution in the loading chamber 12 or the collection vessel 3 into the mixing chamber 11.

Alternatively, the quantity of extraction of the samples in the loading chamber, the quantity of extraction of the solution in the collection vessel 3, and the quantity of extraction of the solution in the mixing chamber 11 can be controlled based on time or flow, which are not limited here.

According to the method for cell collection provided in this embodiment, the secondary separation of the samples is achieved by inputting a pressure in the mixing chamber 11 and by controlling on/off of the communicating structure 4, which greatly simplifies the operation processes and improve the sorting efficiency. During the secondary separation, the samples are not taken out from the collector, thus avoiding secondary contamination of the samples.

Although the present disclosure has been described in detail above with general description, specific embodiments and experiments. However, on this basis, some modifications or improvements can be made to the present disclosure, which is apparent to those skilled in the art. Therefore, these modifications or improvements made without departing from the spirit of the present disclosure shall fall within the protection scope of the present disclosure.

1. A collector comprising:
a housing (1) which is internally provided with a mixing chamber (11) and a loading chamber (12) and which is provided with a filling hole (13) in communication with the mixing chamber (11);
a microfluidic chip (2) arranged in the housing (1); and
a collection vessel (3) detachably connected to the housing (1), wherein a target cell solution separated by the microfluidic chip (2) can flow into the collection vessel (3);
and wherein an inlet of the microfluidic chip (2), the loading chamber (12), and the collection vessel (3) are selectively in communication with the mixing chamber (11) through a communicating structure (4), respectively.

2. The collector according to claim 1, characterized in that, the communicating structure (4) comprises a loading chamber connection hose (41), a chip connection hose (42), and a collection vessel connection hose (43);
the loading chamber (12) is in communication with the mixing chamber (11) through the loading chamber connection hose (41), the inlet of the microfluidic chip (2) is in communication with the mixing chamber (11) through the chip connection hose (42), and the collection vessel (3) is in communication with the mixing chamber (11) through the collection vessel connection hose (43); and
the housing (1) is provided with avoidance ports (5) allowing the loading chamber connection hose (41), the chip connection hose (42), and the collection vessel connection hose (43) to be at least partially exposed outside.

3. The collector according to claim 2, characterized in that, the housing (1) is provided with a chip connection hole (14), one end of the chip connection hole (14) is in communication with a lower end of the mixing chamber (11), and other end of the chip connection hole (14) is connected to the chip connection hose (42).

4. The collector according to claim 2, characterized in that, the housing (1) is provided with a loading chamber connection hole (15) and a collection vessel connection hole (16) near an upper end of the loading chamber (12), the loading chamber connection hose (41) is in communication with the mixing chamber (11) through the loading chamber connection hole (15), and the collection vessel connection hose (43) is connected to the mixing chamber (11) through the collection vessel connection hole (16).

5. The collector according to claim 2, characterized in that, an outer lateral wall of the housing (1) is provided with holding slits (171) configured to respectively hold the loading chamber connection hose (41), the chip connection hose (42) and the collection vessel connection hose (43), and one of the holding slits (171) is in communication with one of the avoidance ports (5).

6. The collector according to claim 5, characterized in that, a lateral wall of the housing (1) comprises an inner plate (17) and a cover plate (18), the holding slit (171) is provided on a side of the inner plate (17) near the cover plate (18), and the avoidance port (5) runs through the cover plate (18).

7. The collector according to claim 1, characterized in that, a holding groove (105) is provided on an outer lateral wall of the housing (1), and the collection vessel (3) is arranged in the holding groove (105).

8. The collector according to claim 1, characterized in that, it further comprises a cover body (7) configured to cover the collection vessel (3), wherein a clamping slot (101) is provided on an outer lateral wall of the housing (1), and the cover body (7) is seated in the clamping slot (101).

9. A cell sorting device comprising the collector according to claim 1.

10. A method for cell collection using the collector according to claim 1, comprising:
communicating the mixing chamber (11) with the microfluidic chip (2) through the communicating structure (4), and inputting a positive pressure into the mixing chamber (11) through the filling hole (13), when it is required to inject a solution in the mixing chamber (11) into the microfluidic chip (2); and
communicating the mixing chamber (11) with the loading chamber (12) or the collection vessel (3) through the communicating structure (4), and inputting a negative pressure into the mixing chamber (11) through the filling hole (13), when it is required to inject the solution in the loading chamber (12) or the collection vessel (3) into the mixing chamber (11).

11. A method for cell collection using the cell sorting device according to claim 9, comprising:
communicating the mixing chamber (11) with the microfluidic chip (2) through the communicating structure (4), and inputting a positive pressure into the mixing chamber (11) through the filling hole (13), when it is required to inject a solution in the mixing chamber (11) into the microfluidic chip (2); and
communicating the mixing chamber (11) with the loading chamber (12) or the collection vessel (3) through the communicating structure (4), and inputting a negative pressure into the mixing chamber (11) through the filling hole (13), when it is required to inject the solution in the loading chamber (12) or the collection vessel (3) into the mixing chamber (11).
